(19) United States
(12) Patent Application Publication
(10) Pub. No.: US 2026/0209166 A1
(43) Pub. Date: Jul. 23, 2026

(54) CONVERSION OF A HYDROCARBON FEEDSTOCK DERIVED FROM OXYGEN-RICH BIOMASS INTO AROMATIC ISOCYANATES

(71) Applicant: IFP Energies Nouvelles, Rueil-Malmaison (FR)

(72) Inventors: Catherine LAROCHE, Rueil-Malmaison Cedex (FR); Ludovic CHAHEN, Rueil-Malmaison Cedex (FR)

(73) Assignee: IFP Energies Nouvelles, Rueil-Malmaison (FR)

(21) Appl. No.: 19/139,572

(22) PCT Filed: Dec. 12, 2023
(86) PCT No.: PCT/EP2023/085339

§ 371 Date: Jun. 16, 2025

(30) Foreign Application Priority Data
Dec. 20, 2022 (FR) FR2213910

(51) Int. Cl. C07C 201/08; C07C 2/74; C07C 4/14; C07C 209/36; C07C 263/00

(52) U.S. Cl. CPC C07C 201/08; C07C 2/74; C07C 4/14; C07C 209/36; C07C 263/00

(57) ABSTRACT

A device for converting an oxygen-rich hydrocarbon-based feedstock obtained from biomass, comprising:
a pyrolysis unit;
a water-gas shift WGS reaction section;
a fractionation train suitable for extracting at least one cut comprising benzene, one cut comprising toluene, one cut comprising xylenes, and one cut comprising aromatic compounds with 9 and 10 carbon atoms;
a nitration reaction section suitable for producing aromatic nitro compounds from at least one cut extracted from said fractionation train;
a reduction reaction section;
an isocyanate synthesis reaction section.

### TECHNICAL FIELD

The invention relates to the conversion of oxygen-rich hydrocarbon-based compounds in the context of producing aromatics for the petrochemical industry. More particularly, the invention relates to the production of chemical compounds of the aromatic isocyanate type, such as diphenylmethylene 4,4′-diisocyanate (MDI) or toluene diisocyanate (TDI), from a single hydrocarbon-based feedstock, preferably of biobased origin.

### PRIOR ART

In general, aromatic isocyanates are produced from:
aromatic compounds derived from petroleum upgrading, such as benzene or toluene; and
phosgene from the conversion of a hydrocarbon-based feedstock (fossil, biomass or other).

Currently, processes for producing aromatic isocyanates use two types of feedstock:
a hydrocarbon-based feedstock which may be converted by gasification into hydrogen (H₂) and carbon monoxide (CO). These two gases can be separated, and the carbon monoxide can then be used to produce phosgene, while the hydrogen can be used as a reducing agent;
an aromatic feedstock, such as benzene or toluene, which successively undergoes a nitrosation step, a reduction step and then a final transformation step into isocyanate using the phosgene produced from the hydrocarbon-based feedstock.

However, phosgene is a toxic and hazardous compound that needs to be handled with care. Finding alternatives to the use of phosgene proves to be beneficial in both environmental and safety terms. The article by Wang et al. (Chinese Journal of Chemistry, 2017, 35, 821-835) lists existing alternatives to the use of phosgene, notably the use of carbon dioxide (CO₂) or carbon monoxide (CO) for the synthesis of isocyanates.

Biomass pyrolysis processes, such as catalytic or thermal pyrolysis processes for instance, allow aromatic hydrocarbons, water and gaseous streams comprising CO and CO₂ to be produced from a single lignocellulosic feedstock. A process based on the pyrolysis of biomass, whether catalytic or thermal, may thus advantageously allow biobased aromatic isocyanates to be synthesized from a single lignocellulosic biomass feedstock and the use of phosgene to be minimized or even avoided.

### OBJECTS OF THE INVENTION

In the preceding context, a first object of the present description is to overcome the problems of the prior art and to upgrade carbon, and in particular biobased carbon in the form of CO and/or CO₂, into high value-added compounds, and in particular aromatic compounds, such as aromatic isocyanates.

Specifically, the present invention relates to a device and a process for maximizing the production of aromatic isocyanates from an oxygen-rich hydrocarbon-based feedstock derived from biomass (e.g. having an elemental oxygen content at least greater than 1% by weight, preferentially at least 3% by weight, very preferentially at least 5% by weight) by converting, at least partly, the CO₂ and optionally the CO, by-products of the pyrolysis of the biomass, into aromatic isocyanate compounds.

A first subject of the invention relates to a device for converting an oxygen-rich hydrocarbon-based feedstock obtained from biomass, comprising:
a pyrolysis unit suitable for producing from a hydrocarbon-based feedstock derived from biomass at least one cut comprising aromatic compounds, a gaseous effluent comprising at least carbon monoxide, and water;
a water-gas shift (WGS) reaction section suitable for producing a gaseous stream comprising hydrogen and carbon dioxide from the carbon monoxide and water coming from the pyrolysis unit;
a fractionation train suitable for extracting at least one cut comprising benzene, one cut comprising toluene, one cut comprising xylenes, and one cut comprising aromatic compounds with 9 and 10 carbon atoms from said cut comprising aromatic compounds;
a nitration reaction section suitable for producing aromatic nitro compounds from at least one cut extracted from said fractionation train;
a reduction reaction section suitable for producing aromatic amines from nitro aromatic compounds and hydrogen from the water-gas shift WGS reaction unit;
an isocyanate synthesis reaction section suitable for producing isocyanates from the aromatic amines from the reduction reaction section, and carbon dioxide from the water-gas shift WGS reaction section, or carbon monoxide from the pyrolysis unit.

According to one or more embodiments according to the invention, said device also comprises a reaction section (8) for separating said gaseous stream (18) comprising hydrogen and carbon dioxide into at least one stream enriched in hydrogen (19) and at least one stream enriched in carbon dioxide (20).

According to one or more embodiments according to the invention, the isocyanate synthesis reaction section (6) is suitable for producing diphenylmethylene 4,4′-diisocyanate (MDI).

According to one or more embodiments according to the invention, the nitration reaction section (3) is suitable for producing nitrobenzene from the cut comprising benzene extracted from said fractionation train (2).

According to one or more embodiments according to the invention, said device also comprises a toluene disproportionation reaction section (11A) suitable for producing a benzene-enriched effluent (23) from the cut comprising toluene from said fractionation train (2).

According to one or more embodiments according to the invention, said device also comprises an aromatic amine condensation reaction section (5) for producing aromatic polyamines from aromatic amines from the reduction reaction section (4) and an aldehyde (21).

According to one or more embodiments according to the invention, the isocyanate synthesis reaction section (6) is suitable for producing toluene diisocyanate (TDI).

According to one or more embodiments according to the invention, the nitration reaction section (3) is suitable for producing dinitrotoluene from the cut comprising toluene extracted from said fractionation train (2).

According to one or more embodiments of the invention, said device also comprises a transalkylation reaction section (11B) suitable for producing a toluene-enriched effluent (25) from the cut comprising benzene and the cut comprising xylene from said fractionation train (2).

Another subject of the invention relates to a process for converting an oxygen-rich hydrocarbon-based feedstock, comprising the following steps:
a) a hydrocarbon-based feedstock originating from biomass (11) is sent to a pyrolysis unit (1) in order to produce at least one cut comprising aromatic compounds (12), a gaseous effluent comprising at least carbon monoxide (9a), and water (10);
b) the carbon monoxide (9a) and the water (10) obtained on conclusion of step a) are sent to a water-gas shift WGS reaction section (7) to produce a gaseous stream (18) comprising hydrogen and carbon dioxide;
c) said cut comprising aromatic compounds (12) obtained on conclusion of step a) is sent to a fractionation train (2) to extract at least one cut comprising benzene, one cut comprising toluene, one cut comprising xylenes, and one cut comprising aromatic compounds with 9 and 10 carbon atoms;
d) at least one extracted cut obtained on conclusion of step c) is sent to a nitration reaction section (3) to produce nitro aromatic compounds (14);
e) said nitro aromatic compounds (14) obtained on conclusion of step d), the hydrogen obtained on conclusion of step b) are sent to a reduction reaction section (4) to produce aromatic amines (15);
f) said aromatic amines (15) obtained on conclusion of step e), and, the carbon dioxide obtained on conclusion of step b), or the carbon monoxide (14) obtained on conclusion of step a) are sent to an isocyanate synthesis reaction section (6) to produce isocyanates (17).

According to one or more embodiments according to the invention, said process also comprises a step g) in which said gaseous stream (18) comprising hydrogen and carbon dioxide obtained on conclusion of step b) is sent to a separation reaction section (8) to obtain at least one stream enriched in hydrogen (19) and at least one stream enriched in carbon dioxide (20).

According to one or more embodiments according to the invention, when diphenylmethylene 4,4′-diisocyanate (MDI) is produced in the isocyanate synthesis reaction section (6), said process also comprises a step h) in which the effluent comprising aromatic compounds (15) is sent to an aromatic amine condensation reaction section (5) to obtain an effluent comprising aromatic polyamines (16).

According to one or more embodiments according to the invention, said process also comprises a step i) in which at least one fraction comprising toluene (22) from the fractionation train (2) is sent to a toluene disproportionation reaction section (11A) to obtain an effluent enriched in benzene and enriched in xylenes (23).

According to one or more embodiments according to the invention, when toluene diisocyanate (TDI) is produced in the isocyanate synthesis reaction section, said process also comprises a step j) in which a stream (24) comprising at least a fraction comprising benzene from the fractionation train (2) and at least a fraction of the xylenes from the fractionation train (2) is sent to a transalkylation reaction section (11B) to obtain a toluene-enriched effluent (25).

### LIST OF FIGURES

FIG. 1 shows a schematic representation of a device of one embodiment according to the present invention allowing aromatic isocyanates to be produced from an oxygen-rich hydrocarbon-based feedstock derived from biomass. In this embodiment, the isocyanates are obtained from the aromatic amines from the reduction reaction section, and either the carbon dioxide from the water-gas shift WGS reaction section or the carbon monoxide from the pyrolysis unit (1).

FIG. 2 shows a schematic representation of a device according to one embodiment according to the present invention allowing 4,4′-diphenylmethylene diisocyanate (MDI) to be produced from an oxygen-rich hydrocarbon-based feedstock derived from biomass.

FIG. 3 shows a schematic representation of a device according to one embodiment according to the present invention allowing toluene diisocyanate (TDI) to be produced from an oxygen-rich hydrocarbon-based feedstock derived from biomass.

### DESCRIPTION OF THE EMBODIMENTS

Embodiments of the device according to the first aspect and of the process according to the second aspect will now be described in detail. In the detailed description that follows, numerous specific details are set out in order to convey a deeper understanding of the device. However, it will be apparent to those skilled in the art that the device can be used without these specific details. In other cases, well-known features have not been described in detail in order to avoid unnecessarily complicating the description.

### Definitions

In the present patent application, the term “to comprise” is synonymous with (means the same thing as) “to include” and “to contain”, and is inclusive or open and does not exclude other elements which are not stated. It is understood that the term “to comprise” includes the exclusive and closed term “to consist of”. Moreover, in the present description, an effluent comprising essentially or solely compounds A corresponds to an effluent comprising at least 90% by weight, preferably at least 95% by weight, very preferably at least 99% by weight, of compounds A.

The term “WGS” (Water Gas Shift) refers to a unit allowing at least partial conversion of CO into CO₂ and thus obtaining a gas enriched in CO₂.

In the present patent application, the groups of chemical elements are given, by default, according to the CAS classification (CRC Handbook of Chemistry and Physics, published by CRC Press, Editor-in-Chief D. R. Lide, 81st edition, 2000-2001). For example, group VIII (or group VIIIB) according to the CAS classification corresponds to the metals from columns 8, 9 and 10 according to the new IUPAC classification; group VIB according to the CAS classification corresponds to the metals from column 6 according to the new IUPAC classification.

### DETAILED DESCRIPTION

The present invention may be defined as a device and a process comprising a sequence of unit operations allowing aromatic isocyanates to be produced from oxygen-rich hydrocarbon-based compounds such as biomass.

One of the features of the present invention may be summarized as the use, at least partly, of the CO₂ produced in a WGS unit from the CO and the water from the biomass pyrolysis unit, and/or at least partly of the CO from the catalytic pyrolysis unit, as a reagent for the synthesis of aromatic isocyanates, such as diphenylmethylene 4,4′-diisocyanate (MDI) or toluene diisocyanate (TDI).

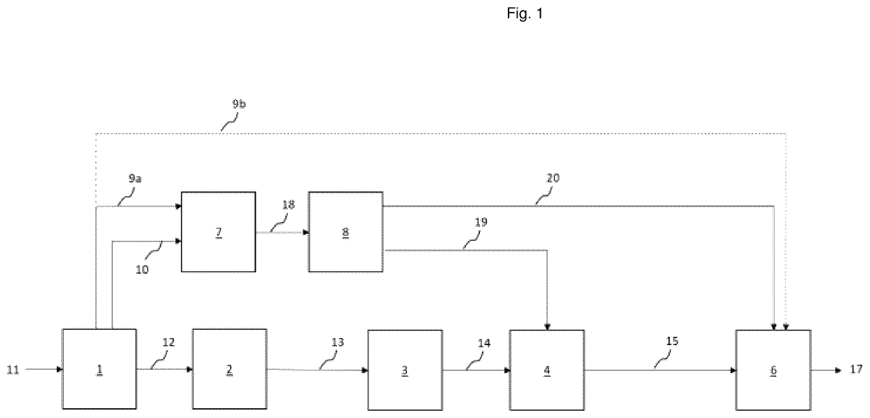

With reference to FIG. 1, according to one or more embodiments, the device for conversion of aromatic compounds comprises:
a pyrolysis unit 1 suitable for producing, from a hydrocarbon-based feedstock derived from biomass 11, at least one cut comprising aromatic compounds 12, a gaseous effluent comprising at least carbon monoxide 9a, and water 10;
a water-gas shift WGS reaction section 7 suitable for producing a gaseous stream 18 comprising hydrogen and carbon dioxide from the carbon monoxide 9a and water 10 coming from the pyrolysis unit 1;
a fractionation train 2 suitable for extracting at least one cut comprising benzene, one cut comprising toluene, one cut comprising xylenes, and one cut comprising aromatic compounds with 9 and 10 carbon atoms from said cut comprising aromatic compounds 12;
a nitration reaction section 3 suitable for producing nitro aromatic compounds 14 from at least one cut extracted from said fractionation train 2;
a reduction reaction section 4 suitable for producing aromatic amines 15 from the nitro aromatic compounds 14 and hydrogen from the water-gas shift WGS reaction unit 7;
an isocyanate synthesis reaction section 6 suitable for producing isocyanates 17 from the aromatic amines 15 from the reduction reaction section 4, and carbon dioxide from the water-gas shift WGS reaction section 7, or carbon monoxide 9b from the pyrolysis unit 1.

With reference to FIG. 1, the pyrolysis unit 1 allows aromatic hydrocarbons and the gases required for the production of aromatic isocyanates to be produced from a hydrocarbon-based feedstock derived from biomass.

According to one or more embodiments, the hydrocarbon-based feedstock sent to the pyrolysis unit 1 is a mixture of hydrocarbon compounds having a content of elemental oxygen at least greater than 1% by weight, preferentially 3% by weight, very preferentially 5% by weight, relative to the total weight of said feedstock. According to one or more embodiments, the hydrocarbon-based feedstock comprises or consists of lignocellulosic biomass or one or more constituents of lignocellulosic biomass chosen from the group formed by cellulose, hemicellulose and lignin.

Lignocellulosic biomass may comprise wood, agricultural waste or plant waste. Other nonlimiting examples of lignocellulosic biomass material are farm residues (straw, corn stover, etc.), forestry residues (products from first thinning), forestry products, dedicated crops (short rotation coppice), agrifood industry residues, organic household waste, waste from woodworking plants, waste construction wood, paper, whether or not recycled.

Lignocellulosic biomass may also come from by-products of the papermaking industry such as Kraft lignin, or black liquors resulting from the manufacture of paper pulp.

According to one or more embodiments, the pyrolysis step (step a) of the process according to the invention) is performed at a temperature of between 400° C. and 1000° C., preferably between 400° C. and 650° C., more preferably between 450° C. and 600° C. and more preferably between 450° C. and 590° C.

The pyrolysis step is also advantageously performed at an absolute pressure of between 0.1 MPa and 0.5 MPa.

In the pyrolysis unit, the hydrocarbon-based feedstock is notably converted at least partially into a pyrolysis effluent comprising hydrocarbon-based compounds with a carbon number ranging from 6 to 10 carbon atoms. The pyrolysis unit also produces a pyrolysis gas comprising CO, CO₂ and H₂, and a liquid by-product fraction.

The products obtained on conclusion of the pyrolysis step are advantageously recovered in the pyrolysis unit in the form of a gaseous effluent comprising BTX.

According to one or more embodiments, the process thus comprises at least one pyrolysis step producing at least one BTX cut (pyrolysis effluent) and a gaseous fraction of incondensables (pyrolysis gas) comprising at least carbon monoxide and carbon dioxide.

With reference to FIG. 1, said gaseous effluent comprising the products obtained on conclusion of the pyrolysis step is then advantageously sent to a fractionation section of the pyrolysis unit so as to separate at least the following cuts:
a gaseous fraction of incondensables 9a and/or 9b, comprising at least carbon monoxide (CO) and carbon dioxide (CO₂);
a liquid cut 12 known as BTX, comprising hydrocarbon-based compounds, the carbon number of which ranges from 6 to 10 carbon atoms;
a liquid cut (not shown in FIG. 1) predominantly comprising compounds having a number of carbon atoms greater than 9, i.e. at least 50% by weight of C9+ compounds; and
water 10.

With reference to FIG. 1, the device according to the invention comprises a Water Gas Shift (WGS) reaction section 7 allowing the production of a gaseous stream 18 comprising at least partly H₂ and CO₂ from H₂O 10 and CO 9a from the pyrolysis unit 1.

In the WGS reaction section 7, part or all of the CO and H₂O produced in pyrolysis unit 1 is fed to at least one WGS reaction reactor to produce a gaseous stream 18 depleted in CO, depleted in H₂O, enriched in CO₂ and enriched in H₂ (step b) of the process according to the invention). This gaseous stream 18 enriched in CO₂ and H₂ may be sent to the CO₂/H₂ separation section 8 to separate the CO₂ and H₂.

The WGS reaction is well known to those skilled in the art (see for example Journal of Catalysis, volume 229, 2005, pages 265-275; and Renewable and Sustainable Energy Reviews, volume 93, 2018, pages 549-565).

According to one or more embodiments, the WGS reaction section 7 comprises at least one reactor used under at least one of the following operating conditions:
temperature of between 250° C. and 500° C., preferentially between 300° C. and 450° C. and even more preferentially between 310° C. and 425° C.;
pressure of between 0.1 and 5 MPa, preferentially between 0.2 and 4 MPa and more preferentially between 0.5 MPa and 3 MPa;
an H₂O/CO mole ratio of between 1 and 4, preferably between 1.5 and 2.5, very preferably between 1.8 and 2.2, such as 2 (±0.1);
a space velocity of the gas entering the reactor of between 1000 and 30 000 mL/gcata/h.

According to one or more embodiments, the reactor of the WGS reaction section is suitable for functioning as a fluidized bed or as a fixed bed.

According to one or more embodiments, the WGS reaction is performed in the presence of a catalyst, such as a catalyst based on transition metals. For example, the catalyst may comprise iron and may optionally be promoted with chromium or copper. According to one or more embodiments, the catalyst comprises at least 50% by weight of Fe₂O₃, preferably at least 65% by weight of Fe₂O₃, relative to the total weight of the catalyst. According to one or more embodiments, the catalyst also comprises between 2% and 20% by weight of Cr₂O₃ and/or CuO, preferably between 5% and 15% by weight of Cr₂O₃ or CuO, relative to the total weight of the catalyst. According to one or more embodiments, the catalyst also comprises between 0.01% and 1% by weight of MgO, preferably between 0.1% and 0.5% by weight of MgO relative to the total weight of the catalyst.

According to one or more embodiments, the WGS reaction section is suitable for producing a WGS gas comprising at least 50% by weight of CO₂ in the mixture of CO, CO₂ and H₂, preferably at least 75% by weight of CO₂, very preferably at least 80% by weight of CO₂.

According to one or more embodiments, the gaseous stream obtained from the pyrolysis unit 1 can be purified before being introduced into the WGS reaction section 7. Purification is directed toward the at least partial removal of the sulfur and nitrogen compounds, halogens, heavy metals, transition metals and unsaturated hydrocarbons of two or more carbons. It may also allow the H₂O and CO composition to be adapted before entering the WGS reaction section, or even to deplete the gas of CO₂. The main technologies for the purification of synthesis gases are: adsorption, absorption, catalytic reactions. In addition to gas purification, it may be necessary to eliminate the fine particles present in the flue gases (technologies: filtration, electrostatic precipitation, cyclones).

With reference to FIG. 1, the device according to the invention may also comprise a CO₂/H₂ separation section 8 allowing CO₂ and H₂ to be separated from the other gases produced in the WGS reaction section 7.

In the CO₂/H₂ separation section 8, part or all of the gaseous stream 18 from the WGS reaction section 7 is treated (optional step g) of the process according to the invention). This step allows at least one CO₂-enriched stream 20 and at least one H₂-enriched stream 19 to be generated, and optionally a gaseous stream comprising the rest of the gases produced (not represented in the figure).

Several separation processes are known to those skilled in the art, such as pressure swing adsorption (PSA), which allows H₂ and CO₂ to be separated from a gas. The adsorbents used in PSA are generally very porous materials with a large specific surface area. Silica gel, active charcoal, activated alumina and zeolites are commonly used and may be used in several consecutive beds. An energy efficient sequencing of the WGS reaction section 7 and the CO₂/H₂ separation section 8 has been described in patent application US 2021/0170322 A. Patent applications US 2018/0036674 A1 and US 2013/0011323 A1 describe examples of high-temperature separation of gases (H₂ and CO₂) dedicated to the treatment of gases exiting a WGS.

With reference to FIG. 1, the device and the process according to the invention (step c) of the process according to the invention) comprise an aromatics fractionation train 2 allowing physical separation of the aromatic compounds coming from the pyrolysis unit 1 and affording at least one effluent comprising aromatic compounds 13.

According to one or more embodiments, the fractionation train comprises several columns for the distillation of aromatic compounds, making it possible to separate the following four cuts:
a cut comprising (e.g. essentially) benzene;
a cut comprising (e.g. essentially) toluene;
a cut comprising (e.g. essentially) xylenes;
a cut comprising (e.g. essentially) aromatic compounds containing 9 and 10 carbon atoms.

In one embodiment according to the invention, when the device and the process according to the invention relate to the production of diphenylmethylene 4,4′-diisocyanate (MDI), the fractionation train 2 is used to obtain an effluent 13 comprising benzene.

In one embodiment according to the invention, when the device and the process according to the invention relate to the production of toluene diisocyanate (TDI), the fractionation train 2 is used to obtain an effluent 13 comprising toluene.

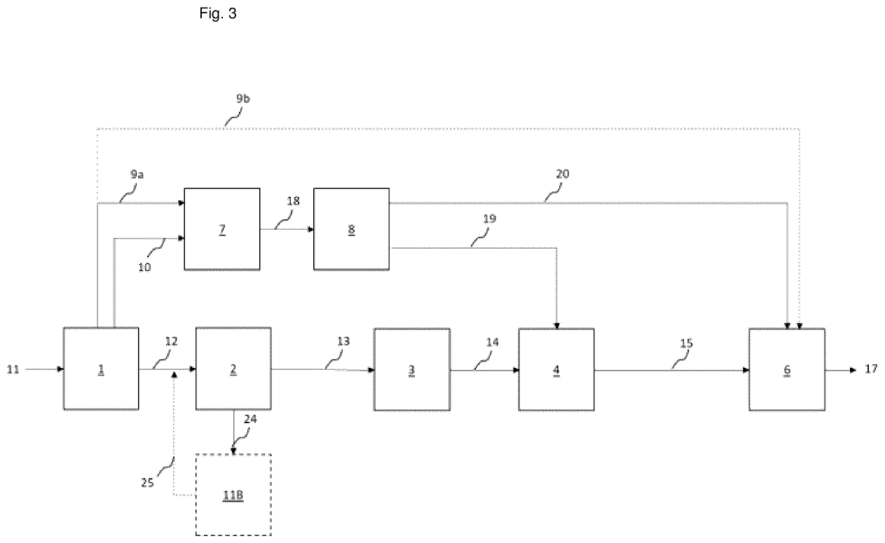

The benzene distillation column is suitable for treating the aromatic cut which is a (e.g. essentially) C6-C10 (A6+) aromatic hydrocarbon-based feedstock, producing at the top the cut comprising benzene which may optionally be sent to the inlet of a transalkylation reaction section 11B (see FIG. 3), and producing at the bottom a (e.g. essentially) C7-C10 (A7+) aromatic effluent.

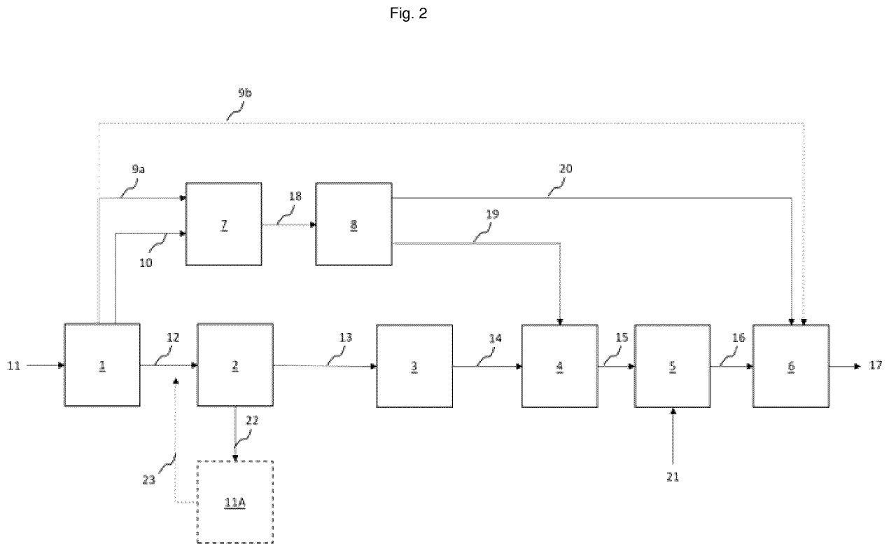

The toluene distillation column is suitable for treating the aromatic C7-C10 (A7+) effluent which is the bottom product of the benzene column, producing at the top the cut comprising toluene which may optionally be directed toward a toluene disproportionation section 11A (see FIG. 2), and producing at the bottom an (e.g. essentially) aromatic C8-C10 (A8+) effluent.

The xylene distillation column is suitable for treating the aromatic cut with 8 or more carbon atoms (A8+) of the aromatic complex feedstock and optionally the bottom effluent from the toluene column, producing at the top the cut predominantly comprising xylenes which may optionally be sent to a transalkylation reaction section 11B (see FIG. 3), and producing at the bottom an effluent (e.g. essentially) comprising C9-C10 (A9+) aromatics.

With reference to FIG. 1, the device and process according to the invention (step d) of the process of the invention) also comprise a nitration reaction section 3 allowing the production of an effluent comprising nitro aromatic compounds 14 from the effluent comprising aromatic compounds 13 from the fractionation train 2.

The nitration step is generally performed in the liquid phase using nitric acid alone or mixed with other strong acids or catalysts and at least one aromatic compound. There are also gaseous routes, notably using NO₂, but these routes are less developed industrially. In the nitration reaction section 3, the effluent comprising the aromatic compounds 13, such as benzene (FIG. 2) or toluene (FIG. 3) coming from the top of the benzene column or the top of the toluene column of the fractionation train 2 is fed to at least one nitration reactor to produce an effluent comprising mononitro or dinitro nitro aromatic compounds 14. This reaction section may also be supplemented with a purification system to enrich the final effluent in mononitro or dinitro aromatic compounds depending on the target. In a preferential manner, the nitration reaction section 3 is used to produce an effluent of nitro aromatic compounds 14 which may be rich in nitrobenzene (according to the embodiment as illustrated in FIG. 2) or in dinitrotoluene (according to the embodiment as illustrated in FIG. 3).

With reference to FIG. 1, the device and process according to the invention (step e) of the process according to the invention) also comprise a reduction reaction section 4 for producing an effluent comprising aromatic amines 15, such as aniline or diaminotoluene, from the effluent comprising the nitro aromatic compounds 14 obtained on conclusion of the nitration reaction section 3 and from the hydrogen obtained on conclusion of the Water Gas Shift (WGS) reaction section 7, optionally from the enriched hydrogen 19 obtained on conclusion of the CO₂/H₂ separation section 8. The reduction of nitro aromatic compounds to aromatic amines is an industrial step well known to those skilled in the art. In the reduction reaction section 4, the nitro aromatic compounds are reduced to the corresponding aromatic amines in the presence of hydrogen and a metal catalyst. The reduction of the nitro aromatic compounds may be performed in the gas phase or in the liquid phase.

When the process is performed in the gas phase, fixed bed or fluidized bed reactors may be used. Palladium or copper, in combination with other metals (lead, vanadium, chromium etc.) on active charcoal or oxide supports are generally used. The average hydrogen/nitro aromatic compound ratio is about 10/1. This step is generally performed at a temperature of between 250° C. and 350° C. and at a pressure of between 0.1 MPa and 1 MPa.

When the process is performed in the liquid phase, nickel-, platinum- and palladium-based catalysts are preferred. In this embodiment, this step is generally performed at a temperature of between 90° C. and 260° C., preferably between 90° C. and 200° C., and at a pressure of between 0.1 MPa and 4 MPa, preferably between 0.1 MPa and 0.6 MPa. More generally, this step is performed at a temperature of between 90° C. and 200° C. and at a pressure of between 0.1 MPa and 0.6 MPa using slurry or fluidized bed reactor technologies.

The reduction reaction section may also be supplemented with a purification system to enrich the final effluent in a specific aromatic amine (dehydration column, distillation, gravimetric separator).

Preferably, the reduction reaction section 4 is used to produce an effluent 15 rich in aniline or toluenediamine (TDA) from benzene or toluene respectively and in the presence of H₂.

With reference to FIG. 1, the device and the process according to the invention (step f) of the process according to the invention) also comprise an isocyanate synthesis reaction section 6 allowing the production of an effluent comprising aromatic isocyanates 17 from the effluent comprising aromatic amines 15.

In one embodiment according to the invention, the effluent comprising the aromatic amines 15 is placed in contact with the CO₂ obtained on conclusion of the WGS reaction section 7, optionally on conclusion of the CO₂/H₂ separation section 8, and in the presence of an alcohol to form, initially, water and N-substituted aromatic carbamates. The N-substituted aromatic carbamates may then be isolated by distillation or via other purification methods. Finally, these compounds undergo thermal cracking to form aromatic isocyanates and reliberate the alcohol, which can be isolated and recycled.

In another embodiment according to the invention, the effluent comprising the aromatic amines 15 is placed in contact with the carbon monoxide (via line 9b) from the pyrolysis unit 1 in the presence of a catalyst, an alcohol and O₂ to form, initially, water and N-substituted aromatic carbamates. The N-substituted aromatic carbamates may then be isolated by distillation or via other purification methods. Finally, these compounds undergo thermal cracking to form aromatic isocyanates and reliberate the alcohol, which can be isolated and recycled.

In another embodiment according to the invention, the carbon monoxide (via line 9b) from the pyrolysis unit is first placed in contact with a chlorine feedstock to produce phosgene, and the phosgene is then placed in contact with the aromatic amines 15. The phosgene synthesis step is generally performed in the gas phase in the presence of a catalyst, generally an active charcoal, at a temperature of between 50° C. and 150° C. In this embodiment, the isocyanate synthesis reaction section 6 comprises a phosgene synthesis unit located upstream of the isocyanate synthesis unit.

FIG. 2 shows a schematic representation of a device and a process according to one embodiment according to the present invention for producing diphenylmethylene 4,4′-diisocyanate (MDI) from an oxygen-rich hydrocarbon-based feedstock derived from biomass.

In this embodiment according to the invention, the device and the process according to the invention (optional step h) of the process according to the invention) also comprise an aromatic amine condensation reaction section 5 for producing an effluent comprising aromatic polyamines 16 from the effluent comprising aromatic compounds 15 from the reduction reaction section 4. In the condensation reaction section 5, the effluent comprising aromatic compounds 15 is placed in contact in the liquid phase with an aldehyde 21 and a strong acid or a solid catalyst to obtain an effluent comprising aromatic polyamines 16. This step is generally performed at a temperature of between 90° C. and 110° C. and at a pressure of between 0.3 and 0.4 MPa.

The condensation reaction section 5 may also be supplemented with a purification system to enrich the final effluent in a specific aromatic polyamine (for example by neutralization or distillation) to avoid pollution such as residual aromatic amines or water.

Preferably, the aniline included in the effluent comprising aromatic compounds 15 reacts with formaldehyde as aldehyde 21 and hydrochloric acid to form diaminodiphenylmethanes and even more preferably 4,4′-diaminodiphenylmethane (MDA).

With reference to FIG. 2, the device and the process according to the invention (optional step i) of the process according to the invention) may also comprise a toluene disproportionation reaction section 11A allowing the production of benzene from the pyrolysis unit 1 to be maximized and thus the production of aromatic isocyanates derived from benzene to be maximized. This unit operation also increases the production of xylenes.

In the toluene disproportionation reaction section 11A, at least one fraction 22 comprising toluene from the top of the toluene column of the fractionation train 2 is fed to at least one disproportionation reactor to produce an effluent enriched in benzene and enriched in xylenes 23. The disproportionation reactor may also be simultaneously fed with H₂. In this case, the benzene-enriched, xylene-enriched and toluene-depleted effluent may undergo a degassing step to release the residual H₂, before being sent to another reaction section of the device according to the invention, and in particular to the reduction reaction section 4. The effluent enriched in benzene and xylenes 23 is sent to the fractionation train 2 at the inlet to the benzene distillation column.

According to one or more embodiments, the disproportionation reaction section 11A comprises at least one disproportionation reactor suitable for use under at least one of the following operating conditions:
temperature of between 200° C. and 600° C., preferentially between 350° C. and 550° C. and even more preferentially between 380° C. and 500° C.;
pressure of between 0.1 MPa and 10 MPa;
WWH of between 0.5 and 5 h⁻¹.

The term WWH corresponds to the weight of hydrocarbon-based feedstock injected hourly, relative to the weight of catalyst charged.

According to one or more embodiments, at least one disproportionation reactor is operated in the presence of a catalyst comprising zeolite, for example mordenite.

According to one or more embodiments, at least one disproportionation reactor is of the fixed bed type.

FIG. 3 illustrates a schematic representation of a device according to one embodiment according to the present invention allowing toluene diisocyanate (TDI) to be produced from an oxygen-rich hydrocarbon-based feedstock derived from biomass.

In this embodiment, the device and the process according to the invention (optional step j) of the process according to the invention) can also comprise a transalkylation reaction section 11B for maximizing the production of toluene from the pyrolysis unit 1 and thus maximizing the production of aromatic isocyanates derived from toluene.

In the transalkylation reaction section 11B, at least one fraction comprising benzene coming from the top of the benzene column of the fractionation train 2 is mixed with the cut comprising xylenes coming from the top of the xylene column of the fractionation train 2, and is fed via line 24 to at least one transalkylation reactor to produce toluene by transalkylation of aromatics lacking methyl groups (i.e, benzene), and with an excess of methyl groups (i.e. xylenes).

The toluene-enriched effluent 25 (relative to the cut comprising benzene and the cut comprising xylenes) from the transalkylation reaction section 11B is sent, in the fractionation train 2, to the inlet of the benzene column, optionally with the aromatic cut.

According to one or more embodiments, the transalkylation reaction section 11B comprises at least one transalkylation reactor which is suitable for use under at least one of the following operating conditions:
temperature of between 200° C. and 600° C., preferentially between 350° C. and 550° C. and even more preferentially between 380° C. and 500° C.;
pressure of between 2 MPa and 10 MPa, preferentially between 2 MPa and 6 MPa and more preferentially between 2 MPa and 4 MPa;
WWH of between 0.5 and 5 h⁻¹, preferentially of between 1 and 4 h⁻¹, and more preferentially of between 2 and 3 h⁻¹.

The term WWH corresponds to the weight of hydrocarbon-based feedstock injected hourly, relative to the weight of catalyst charged.

According to one or more embodiments, the at least one transalkylation reactor is operated in the presence of a catalyst comprising zeolite, for example ZSM-5.

According to one or more embodiments, the at least one transalkylation reactor is of fixed bed type.

Thus, the astute combination between on the one hand a pyrolysis unit 1, a WGS reaction section 7, optionally an H₂/CO₂ separation section 8, a fractionation train 2, a nitration reaction section 3, a reduction reaction section 4, optionally a condensation reaction section 5, and an isocyanate synthesis reaction section 6 allows aromatic isocyanates to be produced from oxygen-rich hydrocarbon-based compounds such as biomass.

### EXAMPLES

Tables 1 and 2 hereinbelow illustrate the percentage gains in carbon from the starting biomass that can be upgraded to MDI (Table 1) and TDI (Table 2) after pyrolysis of 1000 tonnes per day of biomass.

### Example 1 (Non-Compliant): Synthesis of Biobased MDI from Benzene Derived from Pyrolysis of Lignocellulosic Biomass

For a conventional pyrolysis step, 5.6% by weight of the biomass carbon relative to the total weight of the biomass introduced is converted into benzene. For complete conversion of the benzene into MDI, 5.6% by weight of the carbon in the biomass feedstock is upgraded to MDI (see Table 1).

### Example 2 (Inventive): Synthesis of Biobased MDI from Benzene Derived from Pyrolysis of Lignocellulosic Biomass+Upgrading of CO Via WGS

Under the following operating conditions:
all the benzene produced is converted into MDI; and
part of the CO₂ exiting WGS is used to synthesize MDI from MDA; 7% of the carbon in the biomass feedstock is upgraded to MDI (see Table 1).

### Example 3 (Inventive): Synthesis of Biobased MDI from Benzene Derived from Pyrolysis of Lignocellulosic Biomass+Upgrading of CO Via WGS+Disproportionation of Toluene

The disproportionation step allows toluene to be converted into benzene and xylene.

Under the following operating conditions:
all the toluene is converted into xylene and benzene;
all the benzene produced is converted into MDI; and
part of the CO₂ exiting WGS is used to synthesize MDI from MDA; 9.8% of the carbon in the biomass feedstock is upgraded to MDI (see Table 1).

TABLE 1

| | | | | % of C |
|---|---|---|---|---|
| | | | | upgradable to |
| | | % of C | % of C | MDI with WGS |
| | | upgradable | upgradable | and dispropor- |
| | | to MDI | to MDI with | tionation |
| | Pyrolysis | (Example 1: | WGS | of toluene |
| Pyrolysis | output in | non- | Example 2: | (Example 3: |
| products | kmol/h | compliant) | compliant) | compliant) |
| Benzene | 10.8 | 5.6 | 5.6 | 5.6 |
| Toluene | 10.7 | | | 2.8 |
| Ethyl- | 0.3 | | | |
| benzene | | | | |
| Xylene | 7.6 | | | |
| CO₂ | 679.5 | | | |
| CO | 276.4 | | 1.4 | 1.4 |
| Total | | 5.6 | 7.0 | 9.8 |

### Example 4 (Inventive): Synthesis of Biobased TDI from Toluene Derived from Pyrolysis of Lignocellulosic Biomass

For a conventional pyrolysis step, 6.4% by weight of the biomass carbon relative to the total weight of the biomass introduced is converted into toluene. For complete conversion of the toluene into TDI, 6.4% by weight of the carbon in the biomass feedstock is upgraded to TDI (see Table 2).

### Example 5 (Inventive): Synthesis of Biobased TDI from Toluene Derived from Pyrolysis of Lignocellulosic Biomass+Upgrading of CO Via WGS

Under the following operating conditions:
all the toluene produced is converted into TDI;
part of the CO₂ exiting WGS is used to synthesize TDI from MDA; 8.2% of the carbon in the biomass feedstock is upgraded to TDI (see Table 3).

### Example 6 (Inventive): Synthesis of Biobased TDI from Toluene Derived from Pyrolysis of Lignocellulosic Biomass+Upgrading of CO Via WGS+Transalkylation of Benzene and Xylene

Transalkylation allows xylene and benzene to be converted into toluene.

Under the following operating conditions:
all the benzene and all the xylene are converted into toluene
all the toluene produced is converted into TDI
part of the CO₂ exiting WGS is used to synthesize TDI from MDA;
22.5% of the carbon in the biomass feedstock is upgraded to TDI (see Table 2).

TABLE 2

| | | | | % of C |
|---|---|---|---|---|
| | | | | upgradable |
| | | % of C | % of C | to TDI with |
| | | upgradable | upgradable | WGS and |
| | | to TDI | to TDI | benzene/xylene |
| | Pyrolysis | (Example 4: | with WGS | transalkylation |
| Pyrolysis | output in | non- | Example 5: | Example 6: |
| products | kmol/h | compliant) | compliant) | compliant) |
| Benzene | 10.8 | | | 4.8 |
| Toluene | 10.7 | 6.4 | 6.4 | 6.4 |
| Ethylbenzene | 0.3 | | | |
| Xylene | 7.6 | | | 6.3 |
| CO₂ | 679.5 | | | |
| CO | 276.4 | | 1.8 | 5 |
| Total | | 6.4 | 8.2 | 22.5 |

1. A device for converting an oxygen-rich hydrocarbon-based feedstock obtained from biomass, comprising:
a pyrolysis unit (1) suitable for producing, from a hydrocarbon-based feedstock derived from biomass (11), at least one cut comprising aromatic compounds (12), a gaseous effluent comprising at least carbon monoxide (9a), and water (10);
a water-gas shift WGS reaction section (7) suitable for producing a gaseous stream (18) comprising hydrogen and carbon dioxide from the carbon monoxide (9a) and water (10) coming from the pyrolysis unit (1);
a fractionation train (2) suitable for extracting at least one cut comprising benzene, one cut comprising toluene, one cut comprising xylenes, and one cut comprising aromatic compounds with 9 and 10 carbon atoms from said cut comprising aromatic compounds (12);
a nitration reaction section (3) suitable for producing nitro aromatic compounds (14) from at least one cut extracted from said fractionation train (2);
a reduction reaction section (4) suitable for producing aromatic amines (15) from the nitro aromatic compounds (14) and hydrogen from the water-gas shift WGS reaction unit (7); and
an isocyanate synthesis reaction section (6) suitable for producing isocyanates (17) from the aromatic amines (15) from the reduction reaction section (4), and carbon dioxide from the water-gas shift WGS reaction section (7), or carbon monoxide (9b) from the pyrolysis unit (1).

2. The device as claimed in claim 1, further comprising a reaction section (8) for separating said gaseous stream (18) comprising hydrogen and carbon dioxide into at least one stream enriched in hydrogen (19) and at least one stream enriched in carbon dioxide (20).

3. The device as claimed in claim 1, wherein the isocyanate synthesis reaction section (6) is suitable for producing diphenylmethylene 4,4′-diisocyanate (MDI).

4. The device as claimed in claim 3, wherein the nitration reaction section (3) is suitable for producing nitrobenzene from the cut comprising benzene extracted from said fractionation train (2).

5. The device as claimed in claim 3, further comprising a toluene disproportionation reaction section (11A) suitable for producing a benzene-enriched effluent (23) from the cut comprising toluene from said fractionation train (2).

6. The device as claimed in claim 3, further comprising an aromatic amine condensation reaction section (5) for producing aromatic polyamines from aromatic amines from the reduction reaction section (4) and an aldehyde (21).

7. The device as claimed in claim 1, wherein the isocyanate synthesis reaction section (6) is suitable for producing toluene diisocyanate (TDI).

8. The device as claimed in claim 7, wherein the nitration reaction section (3) is suitable for producing dinitrotoluene from the cut comprising toluene extracted from said fractionation train (2).

9. The device as claimed in claim 7, also comprising a transalkylation reaction section (11B) suitable for producing a toluene-enriched effluent (25) from the cut comprising benzene and the cut comprising xylene from said fractionation train (2).

10. A process for converting an oxygen-rich hydrocarbon-based feedstock, comprising the following steps:
a) a hydrocarbon-based feedstock originating from biomass (11) is sent to a pyrolysis unit (1) in order to produce at least one cut comprising aromatic compounds (12), a gaseous effluent comprising at least carbon monoxide (9a), and water (10);
b) the carbon monoxide (9a) and the water (10) obtained on conclusion of step a) are sent to a water-gas shift WGS reaction section (7) to produce a gaseous stream (18) comprising hydrogen and carbon dioxide;
c) said cut comprising aromatic compounds (12) obtained on conclusion of step a) is sent to a fractionation train (2) to extract at least one cut comprising benzene, one cut comprising toluene, one cut comprising xylenes, and one cut comprising aromatic compounds with 9 and 10 carbon atoms;
d) at least one extracted cut obtained on conclusion of step c) is sent to a nitration reaction section (3) to produce nitro aromatic compounds (14);
e) said nitro aromatic compounds (14) obtained on conclusion of step d), the hydrogen obtained on conclusion of step b) are sent to a reduction reaction section (4) to produce aromatic amines (15); and
f) said aromatic amines (15) obtained on conclusion of step e), and, the carbon dioxide obtained on conclusion of step b), or the carbon monoxide (9b) obtained on conclusion of step a) are sent to an isocyanate synthesis reaction section (6) to produce isocyanates (17).

11. The process as claimed in claim 10, further comprising a step g) in which said gaseous stream (18) comprising hydrogen and carbon dioxide obtained on conclusion of step b) is sent to a separation reaction section (8) to obtain at least one stream enriched in hydrogen (19) and at least one stream enriched in carbon dioxide (20).

12. The process as claimed in claim 10, wherein, when diphenylmethylene 4,4′-diisocyanate (MDI) is produced in the isocyanate synthesis reaction section (6), said process further comprises a step h) in which the effluent comprising aromatic compounds (15) is sent to an aromatic amine condensation reaction section (5) to obtain an effluent comprising aromatic polyamines (16).

13. The process as claimed in claim 12, further comprising a step i) in which at least one fraction comprising toluene (22) from the fractionation train (2) is sent to a toluene disproportionation reaction section (11A) to obtain an effluent enriched in benzene and enriched in xylenes (23).

14. The process as claimed in claim 10, wherein, when toluene diisocyanate (TDI) is produced in the isocyanate synthesis reaction section (6), said process further comprises a step j) in which a stream (24) comprising at least a fraction comprising benzene from the fractionation train (2) and at least a fraction of the xylenes from the fractionation train (2) is sent to a transalkylation reaction section (11B) to obtain a toluene-enriched effluent (25).
